(19) United States
(12) Patent Application Publication
(10) Pub. No.: US 2024/0341994 A1
(43) Pub. Date: Oct. 17, 2024

(54) LIMB SUPPORT DEVICE

(71) Applicants: Colleen Rogers, Jenkins Twp, PA (US); Kristen Karlovich, Pittsburgh, PA (US)

(72) Inventors: Colleen Rogers, Jenkins Twp, PA (US); Kristen Karlovich, Pittsburgh, PA (US)

(21) Appl. No.: 18/635,808

(22) Filed: Apr. 15, 2024

Related U.S. Application Data
(60) Provisional application No. 63/459,419, filed on Apr. 14, 2023.

(51) Int. Cl. A61F 5/37

(52) U.S. Cl. CPC A61F 5/37

(57) ABSTRACT

A height-adjustable support for supporting various bodily limbs over a wide range of limb positions, and in hospital rooms, doctors' offices, or other compact environments. The device permits a medical procedure to be performed by a single person while the device supports the limb. The device includes a rigid support body defining a base portion having a lower surface, and a cradle supported above the lower surface, the cradle defining a surface configured to support a limb of a patient. The rigid support body may include multiple discrete block bodies selectively joinable by complementary fasteners, so that the rigid support body is adjustable in height. The cradle may be U-shaped or otherwise concavely-shaped.

### CROSS-REFERENCE TO RELATED APPLICATIONS

This application claims the benefit of priority, under 35 U.S.C. § 119(e), of U.S. provisional patent application No. 63/459,419, filed Apr. 14, 2023, the entire disclosure of each of which is hereby incorporated herein by reference.

### FIELD OF THE INVENTION

The present invention relates generally to limb supports for medical and surgical procedures, and more particularly, relates to a simple, height-adjustable support suitable for use with various different limbs over a range of limb positions, and in hospital rooms or in other compact environments in which space is limited.

### DISCUSSION OF RELATED ART

Various supports are known in the prior art for holding in place the limb of a person. Some of these devices are not height and/or position adjustable, limiting their usability. Others are large and bulky, and unsuitable for use in compact environments in which space is limited. Some of these devices are permanently attached to an operating table or bed, and thus are not mobile, and are positioned for dedicated use to support an arm, or a leg, but not both. Some of these devices have structure limiting how the device can support the limb. Some of these devices are mechanically complex, and expensive.

What is needed is a simple support suitable for use for various different limbs over a range of limb positions, and in hospital rooms or other compact environments in which space is limited.

### SUMMARY

The present invention provides a simple height-adjustable support suitable for use for various different limbs over a range of limb positions, and in hospital rooms, doctors' offices, or other compact environments in which space is limited. In certain embodiments. a limb-supporting cradle portion of the device is height-adjustable, to facilitate use in different contexts and/or for different purposes. The limb support device requires only a single person to use it, and it permits an associated medical procedure to be performed by a single person, thereby eliminating the need for a second person for holding and supporting the limb during a medical procedure, as is commonplace. Further, the device provides several advantages relative to various prior art devices, including that the device is portable and easily transported/moved for use with a patient, and that the device can be used while the patient is in a wheelchair, chair or bed. Further still, use of the device ensures that there is less strain on the patient, and helps the patient to feel more secure and comfortable with the patient's limb in the device, which affords the caregiver ample time to evaluate the limb/wound and/or to perform the medical procedure properly while providing comfort to the patient. Still further, use of the device ensures that there is less strain on caregivers by allowing for adjustment of the height of the cradle, and thereby reducing or eliminating a need for a caregiver to maintain a bent-over position during procedures for a prolonged period of time.

### BRIEF DESCRIPTION OF THE FIGURES

An understanding of the following description will be facilitated by reference to the attached drawings, in which:

FIG. 1 is a front view of a limb support device in accordance with an exemplary embodiment of the present invention;

FIG. 2 is a side view of the limb support device of FIG. 1;

FIG. 3 is a perspective view of the limb support device of FIG. 1;

FIG. 4 is a front view of an alternative limb support device in accordance with another exemplary embodiment of the present invention, shown in an exploded view;

FIG. 5 is a side view of the limb support device of FIG. 4;

FIG. 6 is a perspective view of the limb support device of FIG. 4;

FIG. 7 is a perspective view of the limb support device of FIG. 4, shown in a partially assembled state;

FIG. 8 is a perspective view of the limb support device of FIG. 4, shown in a fully assembled state;

FIG. 9 is a side view of the limb support of FIGS. 1 and 5, shown positioned to make a leg/foot accessible for treatment of a leg/foot wound;

FIG. 10 is a side view of the limb support of FIG. 7, shown positioned to make a leg/foot accessible for treatment of a leg/foot wound; and

FIG. 11 is a side view of the limb support of FIG. 8, shown positioned to make a hand/arm accessible for treatment of a hand/arm wound.

### DETAILED DESCRIPTION

The present invention provides a simple, portable support, that may be height-adjustable, and that is suitable for use for various different limbs over a range of limb positions, and in hospital rooms, doctors' offices, or other compact environments in which space is limited. Such a device requires only a single person to use/operate it, and it permits an associated medical procedure to be performed by a single person, thereby eliminating the need for a second person for holding and supporting the limb during a medical procedure, as is commonplace. Further, the device provides several advantages relative to various prior art devices, including that the device is portable and easily transported/moved to a suitable position in relation to the patient, and that the device can be used while the patient is in a wheelchair, chair or bed. Further still, use of the device ensures that there is less strain on the patient, and helps the patient to feel more secure and comfortable with the patient's limb in the device, which affords the caregiver ample time to evaluate the limb/wound and/or to perform the medical procedure properly while providing comfort to the patient. Still further, use of the device ensures that there is less strain on caregivers by allowing for adjustment of the height of the cradle, and thereby reducing or eliminating a need for a caregiver to maintain a bent-over position during procedures for a prolonged period of time.

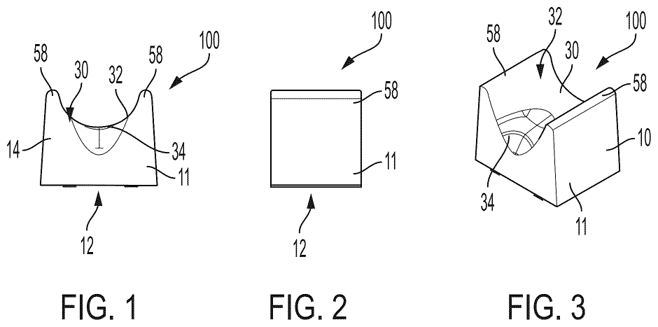

Referring now to FIGS. 1-3, an exemplary limb support device 100 in accordance with a first exemplary embodiment of the present invention is shown. In this embodiment, the limb support device 100 is constructed as a fixed-height body. In this exemplary embodiment, the limb support device 100 is constructed as a unitary body. As will be noted from FIG. 1, the exemplary limb support device 100 is a free-standing device separate and apart from any hospital bed.

As will be appreciated from FIG. 1, the limb support device 100 includes a base portion 11 having an upwardly extending body 14 supporting a cradle 30. The base portion 11 has a lower surface 16, which may be placed on a floor, bed, etc. to support the device as described further herein.

The cradle 30 is preferably generally concave in shape (open upwardly), and may be U-shaped, and oriented with the open end of the “U” facing upwardly, to facilitate receipt and support of a patient's limb, as shown in FIGS. 1-3. The cradle 30 is preferably elongated longitudinally, in the same direction of elongation as the base 10, as best shown in FIGS. 1 and 3, and may have a uniform or substantially uniform cross-section along its length, as shown in FIG. 1.

The cradle 30 may be defined by a first concavity 32 at least partially defined by at least one ridge 38. The first concavity 32 of the cradle 30 is dimensioned to accommodate a broad range of human leg limb (e.g., such as a thigh or calf) sizes. The first concavity 32 may be defined such that an axis of elongation of the cradle is parallel to a plane of the lower surface 16, as shown in FIGS. 1-3.

In this exemplary embodiment, the cradle 30 is further defined by a second concavity 34 smaller in dimensions that the first concavity 32. The second concavity 34 of the cradle 30 is dimensioned to accommodate a broad range of human arm limb (e.g., such as a forearm) sizes. The second concavity 34 may be defined such that an axis of elongation of the cradle is transverse to a plane of the lower surface 16, as shown in FIGS. 1-3.

Together, the two differently-oriented concavities facilitate the supporting of a limb (either an arm or leg) in different orientations/angles, to better allow for supporting of a limb in a manner that is comfortable for a patient. For example, in certain positions of the patient, supporting the leg in the second concavity may be more comfortable than supporting the leg in the first concavity, etc. Accordingly, the first concavity may be positioned to have a first angular orientation (e.g., relative to an axis of elongation of the device), and the second concavity may be positioned to have a second angular orientation different from the first angular orientation.

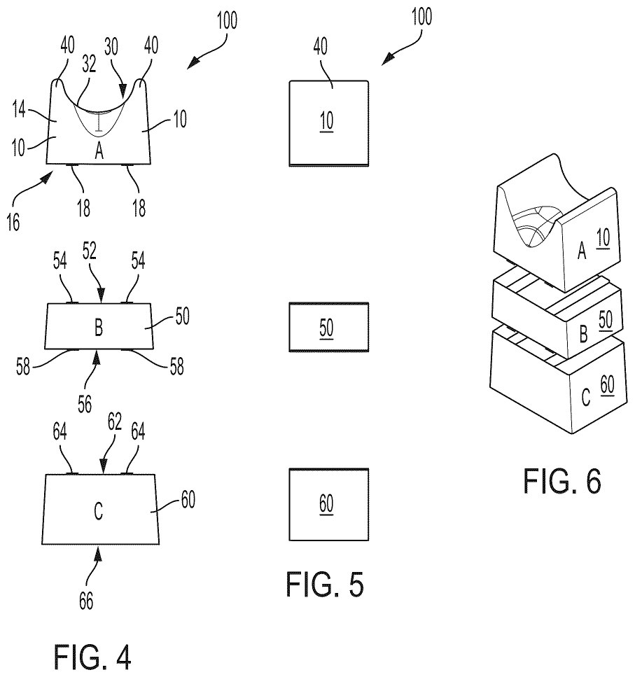

Referring now to FIGS. 4-6, an alternative exemplary limb support device 100 in accordance with a second exemplary embodiment of the present invention is shown. In this embodiment, the limb support device 100 is constructed to have a rigid support body including multiple fixed-height bodies (e.g., a top block A (10), a middle block B (50), and bottom block C (60) that may be selectively combined to provide height-adjustability to the cradle. In this manner, the rigid support body is effective a support that is adjustable in length, by selectively mating individual block bodies. In this exemplary embodiment, each body of the limb support device 100 is constructed as a unitary body.

As will be appreciated from FIGS. 4-6, the top block 10 (A) of the limb support device 100 is similar to that described above. Accordingly, top block A includes a base portion 11 having an upwardly extending body 14 supporting a similar cradle 30. The base has a lower surface 16, which may be placed on a floor, bed, etc. to support the device as described further herein. Further, in this embodiment, the lower surface 16 of the base 10 is provided with a connector 18 operable to form a readily-releasable mechanical interconnection with another block (e.g., middle block B (50) and/or bottom block C (60)), for the purposes described herein. In this exemplary embodiment, the connector 18 is provided in the form of two strips 18a, 18b of one of hook fastener and look fastener of a hook-and-loop fastener system.

In this embodiment, the limb support device includes a middle block B 50 and a bottom block C 60, for illustrative purposes, although any suitable number of blocks may be employed. The primary function of each additional bloc 50, 60 is to be joinable beneath the top block 10, to support the top block 10 and its cradle at a different height above a floor, etc., by virtue of the height of the additional blocks 50, 60. Each block 50, 60 may be generally similar to the top block A 10, except that the other blocks 50, 60, may omit the cradle/ridge portions included in the top block, for the purposes described herein.

In this exemplary embodiment, the connector 18 supported on the lower surface 16 of the top block 10 is provided in the form of two strips 18a, 18b of one of hook fastener and look fastener of a hook-and-loop fastener system. Accordingly, a complementary connector 54 complementary to the connector 18 on the bottom surface 16 of the top block 10 is provided on a top surface 52 of the middle block B 50. This allows the top block A 10 to be readily and easily matable/releasable/rematable from the middle block 50B, as best shown in FIG. 4.

Accordingly, a second complementary connector 64 complementary to the connector 18 on the bottom surface 16 of the top block 10 is provided on a top surface 62 of the bottom block C 60. This allows the top block A 10 to be readily and easily matable/releasable/rematable from the bottom block 60 C, as best shown in FIG. 4.

Additionally, a second connector 58 complementary to the connector 64 on the top surface 62 of the bottom block C 60 is provided on a bottom surface 56 of the bottom block C 60. This allows the top block A 10 to be readily and easily matable/releasable/rematable from the bottom block 60 C, as best shown in FIG. 4.

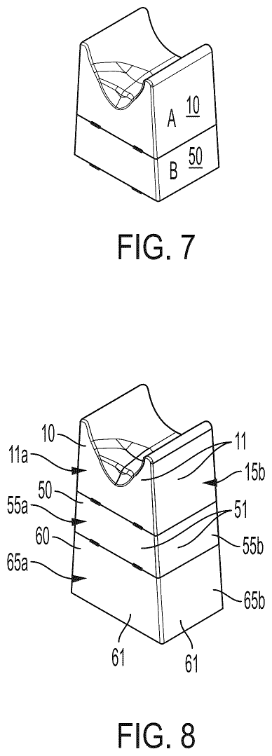

Accordingly, it should be appreciated that block A 10 can be used alone to provide a cradle 30 positioned at a first height (e.g., as shown in FIG. 3). Additionally, block A 10 and block B 50 can be joined by their complementary connectors to provide a cradle 30 positioned at a second height (e.g., as shown in FIG. 7). Further, block A 10, block B 50 and block C 60 can be joined by their complementary connectors to provide a cradle 30 positioned at a third height (e.g., as shown in FIG. 8).

In certain embodiments, the block B 50 and block C may have different heights between their respective bottom and top surfaces. In such cases, the cradle is further height-adjustable by joining block A 10 and block C 60, to provide for 4 height combinations from 3 blocks.

In certain embodiments, the sidewalls 15a/15b/15c/15d, 55a/55b/55c/55d, 65a/65b/65c/65d of the blocks may not be parallel, such that the walls are angled away from parallel, and the blocks and their sidewalls may be configured so that when assembled in a particular fashion (e.g., block A 10 on top of block B 50, and block B 50 on top of block C 60, as shown in FIG. 8) the corresponding sidewalls of multiple blocks (e.g., 15a, 55a, 65a) align in a plane, as shown in FIG. 8.

In certain embodiments, a base portion 11, 51, 61 of each block 10, 50, 60 is has a larger cross-sectional area that of its respective upper portion, such that the block tapers away from the base portion 11, 51, 61, as shown in FIG. 8. This provides a relatively broader base for each block or assembly of blocks that contributes to overall positional stability of the block/assembly/device 100 during use, by resisting toppling of the device 100.

The base 10, body 16 and cradle/ridge 30/38 are constructed to be relatively rigid (sufficient to support limbs) unitary body formed of a lightweight material, such as ILD polyurethane foam. In embodiments that include them, additional blocks (such as blocks B 50 and C 60 may be similarly formed.

Accordingly, the blocks act as adjustable support(s) providing for adjustment of a base portion of the cradle 30, e.g., from about 16″ above the floor or bed (e.g., for supporting the foot of a patient seated in a wheelchair) to about 48″ above the floor (e.g., a comfortable height for a standing nurse and a patient in an elevated bed), or within any sub-range thereof, to allow for accommodation of a limb at a range of heights likely to be encountered as the patients sits in a chair or lies in a hospital bed.

Accordingly, a single instance of the limb support device 100 is movable/portable, and positionable as desired as supported on a hospital bed, table, chair, etc., and on a floor, separately/independently from a hospital bed. table, chair, etc., for use to support various different limbs of a person/patient, such as a left arm, right arm, left leg or right leg, at different heights, locations, orientations, etc., as desired.

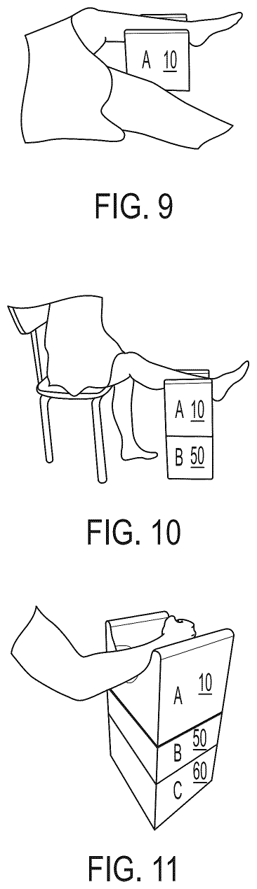

For illustrative purposes, FIG. 9 shows the limb support device 100 of FIGS. 4-8 (or FIG. 1) as used in/supported on a hospital bed, in position for use to support a leg/foot of a patient laying in the bed, which is shown supported in the cradle 30 of the block A 10.

For further illustrative purposes, FIG. 10 shows the limb support device of FIGS. 4-8 in a partially assembled state (block A 1A and block B 50 joined by their connectors), supported on a floor adjacent a chair, in position for use to support a leg/foot of a patient sitting in the chair, which is shown supported in the cradle 30 of the block A 10.

For still further illustrative purposes, FIG. 11 shows the limb support device of FIGS. 4-8 in a fully assembled state (block A 10, block B 50 and block C 60 joined by their connectors), supported on a floor or table, in position for use to support an arm/had of a patient sitting/standing near the limb support device 100.

In use, the limb support device 100 may be assembled as needed by mating/interlocking mating connectors of one or more blocks A 10, B 50, C 60, etc. to support the cradle 30 of block A 10 at a desired height about a support surface on which the device 100 will rest during an intended procedure, e.g., on or adjacent a bed, chair, etc. in which the patient is resting.

After the device is assembled placed into an operative position as desired, a patient's limb may be placed in the cradle 30. This may involve orienting the device 100 such that the patient's limb rests comfortably against the cradle 30, e.g., in relation to the first concavity 32 and/or second concavity 34. Comfort may be obtained by rotating the device 100 about is longitudinal axis, so that the limb either is or is not in contact with the second concavity 34, for many patient limb positions, due to the asymmetric concavity design relative to the front end and back end of the cradle.

In this manner, the patient's limb may be reliably supported by the limb support device 100 in a handsfree fashion, freeing the hands of a healthcare provider to perform a medical/surgical procedure, and without requiring assistance of another healthcare provider to support the limb during the medical/surgical procedure. After the procedure has been completed, device may be disassembled, if needed, by separating the mating connectors, and the blocks may be cleaned by wiping them with a suitable sanitation product of a type conventionally used in hospital/medical/clinical settings, to prepare the device for subsequent use with a different person.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A limb support device comprising:
a rigid support body defining a base portion having a lower surface;
a cradle supported on said rigid body, said cradle being generally concave in shape and defining a surface configured to support a limb of a patient, said cradle being supportable at more than one height position relative to said lower surface.

2. The limb support device of claim 1, wherein said rigid support body comprises:
a first block body having a lower surface;
a first connector supported on said lower surface of said first block body;
a second block body having an upper surface; and
a mating connector, complementary to said first connector, supported on said upper surface of said second block body.

3. The limb support device of claim 2, wherein said lower surface of said rigid support body is defined on said second block body.

4. The limb support device of claim 1, wherein said rigid support body comprises:
a first block body having a first lower surface;
a first connector supported on said first lower surface of said first block body;
a second block body having an upper surface and a second lower surface;
a first mating connector, complementary to said first connector, supported on said upper surface of said second block body;
a second connector supported on said second lower surface of second first block body;
a third block body having a second upper surface and a second lower surface; and
a second mating connector, complementary to said second connector, supported on said second upper surface of said third block body.

5. The limb support device of claim 4, wherein said lower surface of said rigid support body is defined on one of said second block body and said third block body.

6. The limb support device of claim 4, wherein said first connector and said second connector are similar in configuration, and wherein said first mating connector and said second mating connector are similar in configuration.

7. The limb support device of claim 4, wherein said first connector and said second connector comprise one of hook fasteners and loop fasteners of a hook-and-loop fastener system, and wherein said first mating connector and said second mating connector comprise another of hook fasteners and loop fasteners of the hook-and-loop fastener system.

8. The limb support device of claim 4, wherein said second block body defines a first height between said upper surface and said second lower surface, and wherein said third block body defines a second height between said second upper surface and said third lower surface that is different from said first height.

9. The limb support device of claim 2, wherein each block body defines at least one sidewall that tapers away from a respective lower surface of each respective block body.

10. The limb support device of claim 10, wherein respective sidewalls of at least two block bodies align in a plane when the block bodies are mated by said connector and mating connector.

11. The limb support device of claim 1, wherein said rigid body comprises at least two block bodies, each of said at least two block bodies being constructed as a unitary body.

12. The limb support device of claim 1, wherein both said rigid support body and said cradle extend in a longitudinal direction of elongation.

13. The limb support device of claim 1, wherein said cradle is defined by a first concavity at least partially defined by at least one ridge, said first concavity being dimensioned to accommodate a range of human limb sizes.

14. The limb support device of claim 13, wherein said first concavity is defined to have an axis of elongation extending parallel to a plane of said lower surface.

15. The limb support device of claim 13, wherein said first concavity is dimensioned to accommodate a range of sizes of human leg limbs, and wherein said cradle is further defined by a second concavity dimensioned to accommodate a range of sizes of human arm limbs.

16. The limb support device of claim 15, wherein said second concavity is defined to have a second axis of elongation that is transverse to a plane of said lower surface.

17. The limb support device of claim 13, wherein said first concavity is positioned to have a first angular orientation, and wherein said second concavity is positioned to have a second angular orientation different from said first angular orientation.

18. A limb support device comprising:
a rigid support body comprising:
a first block body having a first lower surface;
a first connector supported on said first lower surface of said first block body;
a second block body having an upper surface and a second lower surface;
a first mating connector, complementary to said first connector, supported on said upper surface of said second block body;
a second connector supported on said second lower surface of second first block body;
a third block body having a second upper surface and a second lower surface; and
a second mating connector, complementary to said second connector, supported on said second upper surface of said third block body; and
a cradle defined by said first block body, said cradle being generally concave in shape and defining a surface configured to support a limb of a patient, said cradle being supportable at more than one height position relative to said lower surface.

19. The limb support device of claim 18, wherein said second block body defines a first height between said upper surface and said second lower surface, and wherein said third block body defines a second height between said second upper surface and said third lower surface that is different from said first height.

20. The limb support device of claim 18, wherein said cradle is defined by:
a first concavity at least partially defined by at least one ridge, said first concavity being dimensioned to accommodate a human limb; and
a second concavity dimensioned to accommodate the human limb.

21. The limb support device of claim 20, wherein said first concavity is positioned to have a first angular orientation, and wherein said second concavity is positioned to have a second angular orientation different from said first angular orientation.
